SEQUENCE LISTING (10 sequences)

SEQ ID NO: 1 - protein, 683 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 2 - DNA, 2691 bp, Homo sapiens
gcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctcctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 3 - protein, 585 aa, Homo sapiens
  PEPTIDE (1)..(585): 69 to 653 amino acid sequence of human ID No.1
YQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEI

SEQ ID NO: 4 - DNA, 1857 bp, Mouse Intracisternal A-particle
gcaggtcccgccaagtcaccctaccagctggtgctgcagcatagccggctccggggtcgccagcacggccccaatgtatgtgctgtgcagaaggtcattggcaccaacaagaaatacttcaccaactgcaagcagtggtaccagaggaagatctgcggcaagtcgacagtcatcagttatgagtgctgtcctggatatgaaaaggtcccaggagagaaaggttgcccagcagctcttccgctctcaaatctgtatgagaccatgggagttgtgggatcgaccaccacacagctgtatacagaccgcacagaaaagctgaggcctgagatggagggacccggaagcttcaccatctttgctcctagcaatgaggcctggtcttccttgcctgcggaagtgctggactccctggtgagcaacgtcaacatcgaactgctcaatgctctccgctaccacatggtggacaggcgggtcctgaccgatgagctcaagcacggcatgaccctcacctccatgtaccagaattccaacatccagatccatcactatcccaatgggattgtaactgttaactgtgcccggctgctgaaggctgaccaccatgcgaccaacggcgtggtgcatctcattgacaaggtcatttccaccatcaccaacaacatccagcagatcattgaaatcgaggacacctttgagacacttcgggccgccgtggctgcatcaggactcaataccgtgctggagggcgacggccagttcacactcttggccccaaccaacgaggcctttgagaagatccctgccgagaccttgaaccgcatcctgggtgacccagaggcactgagagacctgctaaacaaccacatcctgaagtcagccatgtgtgctgaggccattgtagctggaatgtccatggagaccctggggggcaccacactggaggtgggctgcagtggggacaagctcaccatcaacgggaaggctgtcatctccaacaaagacatcctggccaccaacggtgtcattcatttcattgatgagctgcttatcccagattcagccaagacactgcttgagctggctggggaatctgacgtctccactgccattgacatcctcaaacaagctggcctcgatactcatctctctgggaaagaacagttgaccttcctggcccccctgaattctgtgttcaaagatggtgtccctcgcatcgacgcccagatgaagactttgcttctgaaccacatggtcaaagaacagttggcctccaagtatctgtactctggacagacactggacacgctgggtggcaaaaagctgcgagtctttgtttatcgaaatagcctctgcattgaaaacagctgcattgctgcccatgataagaggggacggtttgggaccctgttcaccatggaccggatgttgacacccccaatggggacagttatggatgtcctgaagggagacaatcgttttagcatgctggtggccgccatccagtctgcaggactcatggagatcctcaaccgggaaggggtctacactgtttttgctcccaccaatgaagcgttccaagccatgcctccagaagaactgaacaaactcttggcaaatgccaaggaacttaccaacatcctgaagtaccacattggtgatgaaatcctggttagcggaggcatcggggccctggtgcggctgaagtctctccaaggggacaaactggaagtcagctcgaaaaacaatgtagtgagtgtcaataaggagcctgttgccgaaaccgacatcatggccacaaacggtgtggtctatgccatcaacactgttctgcagccgccagccaaccgaccacaagaa

SEQ ID NO: 5 - protein, 609 aa, Mouse Intracisternal A-particle
  PEPTIDE (1)..(609): 23 to 641 amino acid sequence of mouse
AGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNV

SEQ ID NO: 6 - DNA, 391 bp, Artificial Sequence
  aig-h3 D-IV
gtttgggaccctgttcaccatggaccggatgttgacacccccaatggggacagttatggatgtcctgaagggagacaatcgttttagcatgctggtggccgccatccagtctgcaggactcatggagatcctcaaccgggaaggggtctacactgtttttgctcccaccaatgaagcgttccaagccatgcctccagaagaactgaacaaactcttggcaaatgccaaggaacttaccaacatcctgaagtaccacattggtgatgaaatcctggttagcggaggcatcggggccctggtgcggctgaagtctctccaaggggacaaactggaagtcagctcgaaaaacaatgtagtgagtgtcaataaggagcctgttgccgaaaccgac

SEQ ID NO: 7 - protein, 140 aa, Artificial Sequence
  aig-h3 D-IV(1X) amino acid sequence
LTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPAN

SEQ ID NO: 8 - protein, 280 aa, Artificial Sequence
  aig-h3 D-IV(2X) amino acid sequence
LTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPAN

SEQ ID NO: 9 - protein, 420 aa, Artificial Sequence
  aig-h3 D-IV(3X) amino acid sequence
LTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPAN

SEQ ID NO: 10 - protein, 560 aa, Artificial Sequence
  aig-h3 D-IV(4X) amino acid sequence
LTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPAN

(19) United States
(12) Patent Application Publication
(10) Pub. No.: US 2005/0164197 A1
(43) Pub. Date: Jul. 28, 2005

(54) Method for measuring the amount of betaig-h3 protein and diagnostic kit using the same

(76) Inventors: In-San Kim, Soosung-ku (KR); Jong-Sub Bae, Taegu (KR)

Correspondence Address: LICATLA & TYRRELL P.C., 66 E. MAIN STREET, MARLTON, NJ, 08053, US

(21) Appl. No.: 10/511,719

(22) PCT Filed: Oct. 22, 2002
(86) PCT No.: PCT/KR02/01975

(30) Foreign Application Priority Data
Apr. 19, 2002 (KR) 2002/21488

(51) Int. Cl. C12Q 1/68; G01N 33/53

(52) U.S. Cl. 435/6; 435/7.1

(57) ABSTRACT

The present invention relates to the method for measuring the amount of βig-h3 protein and diagnostic kit using the same. Particularly, it relates to the method for measuring the amount of βig-h3 protein in the body fluids by specific binding reaction between βig-h3 protein or recombinant proteins of fas-1 domain in the βig-h3 protein (including their fragments or their derivatives) and their ligands and relates to diagnostic kit for the renal diseases, hepatic diseases, rheumatoid arthritis or cardiovascular diseases comprising βig-h3 protein or recombinant proteins of fas-1 domain in the βig-h3 protein (including their fragments or their derivatives) and their ligands. The method and kit of the present invention can be effectively used as sensitive diagnostic method for the extent of damage or progress of the renal diseases, hepatic diseases, rheumatoid arthritis or cardiovascular diseases.

### FIELD OF THE INVENTION

The present invention relates to a method for measuring the amount of βig-h3 protein and diagnostic kit using the same. Particularly, it relates to a method for measuring the amount of βig-h3 protein in the body fluids by specific binding reaction between βig-h3, protein or recombinant proteins of fas-1 domain in the βig-h3 protein (including their fragments or their derivatives) and their ligands and relates to diagnostic kit for the renal diseases, hepatic diseases, rheumatoid arthritis or cardiovascular diseases comprising βig-h3 protein or recombinant proteins of fas-1 domain in the βig-h3 protein (including their fragments or their derivatives) and their ligands.

### BACKGROUND ART OF THE INVENTION

βig-h3 is an extracellular matrix protein induced by TGF-β in many kinds of cells including human melanoma cells, mammary ephithelial cells, keratinocytes and lung fibroblasts. TGF-β (transforming growth factor-β) is involved in the growth and differentiation of many kinds of cells and the mammals have three kinds of TGF-β (TGF-β1, TGF-β2 and TGF-β3). The TGF-β has been known to have many sophisticated functions such as growth control, immune response regulation, stimulating bone-formation, inducing cartilage specific macromolecule, stimulating the wounding healing, etc (Bennett, N. T. et al., Am. J. Surg. 1993, 165, 728). TGF-α is expressed in epithelial cells during wound-healing, probably in order to stimulate the expression of integrin in keratinocytes during the regeneration of epithelial cells. Recent studies on TGF-β expression disclosed that TGF-β3 mRNA is expressed both in epithelia of normal skin and in epithelia under recovery from acute or chronic wounds while TGF-β1 mRNA is expressed only in regenerated epithelia from acute wounds and TGF-β2 mRNA is not expressed at all (Schmid, P. et al., J. Pathol., 1993, 171, 191). Though the concrete theory on the mechanism of the above has not been established yet, TGF-β is believed to play a key role in regeneration of epithelia.

βig-h3, a TGF-β induced gene h3, was first found by Stonier et al. Precisely, the βig-h3 was found during the search of cDNA library differential screening data from A549 cell line, a human lung adenocarcinoma cell line treated with TGF-β1 and it was reported that βig-h3 was 20-fold increased 2 days after TGF-β1 treatment (Stonier, C. et al., DNA cell Biol., 1992, 11, 511). It was also confirmed by DNA sequencing that βig-h3 is composed of 683 amino acids represented by SEQ. ID. No 1 having amino-terminal secretory sequence and carboxy-terminal Arg-Gly-Asp(RGD) enabling ligand recognition against some integrins.

βig-h3 contains 4 homogeneous internal repeated domains along with RGD motif, which are observed in membrane proteins or secretory proteins of mammals, insects, sea urchin, plants, yeasts and bacteria, etc in a state of well-preserved sequence. Proteins such as periostin, fasciclin I, sea urchin HLC-2, algal-CAM and mycobacterium MPB70 also contain the above preservative sequence (Kawamoto, T. et al., Biochem. Biophys. Acta., 1998, 1395, 288). The homogeneous domain (referred as “fas-1 domain” hereinafter) preserved well in those proteins is composed of 110-140 amino acids containing two very preservative branches (H1 and H2) composed of 10 amino acids each. βig-h3, periostin and fasciclin I have 4 fas-1 domains, HCL-2 has 2 and MPB70 has only 1 fas-1 domain. Some of those proteins, as cell adhesion molecules, are known to intermediate the attachment and the detachment of cells although the biological functions of those proteins are not been fully explained yet. For example, βig-h3, periostin and fasciclin I intervene the attachment of fibroblasts, osteoblasts and nerve cells, respectively and algal-CAM is confirmed to be a cell adhesion molecule residing in embryos of volvox (LeBaron, R. G. et al., J. Invest. Dermatol., 104, 844, 1995; Horiuchi, K. et al., J. Bone Miner. Res., 1999, 14, 1239; Huber, O. et al., EMBO J., 1994, 13, 4212).

A purified βig-h3 protein stimulates adhesion and spread of fibroblasts of skin but obstructs adhesion of A549, HeLa and WI-38 cells in serum-free medium. Especially, the βig-h3 obstructs tumor cell growth, colony formation and appearance. In fact, tumor cell growth in nude mouse prepared by transfecting Chinase hamster ovary cells with βig-h3 expression vector was remarkably decreased, which was clearly stated in U.S. Pat. No. 5,714,588 and No. 5,599,788. In addition, a method for stimulating spread and adhesion of fibroblasts around the wounded area by contacting required amount of βig-h3 with the wound was also stated in those patents. Therefore, as a cell adhesion molecule highly induced by TGF-§ in many cells, βig-h3 plays an important role in cell growth, cell differention, wound healing, morphogenesis and cell adhesion.

Although βig-h3 is an effective useful material, it is not fully supplied since only the minimum βig-h3 is generated in human body. In order to solve this problem, a method to prepare βig-h3 by expressing it in eukaryotic cell system using genetic engineering was developed. In that case, though, the growth of cells producing βig-h3 was much slower than that of other cells, resulting in difficulty in obtaining enough amount of βig-h3 producing cells. Therefore, the present inventors established a purification method with which mass-expression of recombinant proteins containing whole βig-h3 protein or some of its domains was possible using E. coli as a host, confirmed that those recombinant proteins supported cell adhesion and spread, and applied for a patent (Korea patent Application #2000-25664).

Cell adhesion activity of βig-h3, a cell adhesion molecule, was first reported in human dermal fibroblasts and then disclosed in chondrocytes, peritoneal fibroblasts and human MRC5 fibroblasts as well. Cell adhesion activity of βig-h3 was thought to be mediated by RGD motif residing in carboxyl terminal of βig-h3 in the early days. But it was reported later that RGD motif was not required for stimulating the spread of chondrocytes and a mature βig-h3 in which RGD motif was deficient by carboxyl-terminus processing could hinder cell adhesion. Resultingly, it was confirmed that RGD motif was not an indispensable mediator for cell adhesion activity of βig-h3. Recent studies have further confirmed that βig-h3 stimulates cell adhesion and spread, especially the spread of fibroblasts, by working with integrin α 1β1 independently while RGD motif of βig-h3 is not required for cell spread mediated by βig-h3 (Ohno, S., et al., Biochim. Biophys. Acta, 1999, 1451, 196). Besides, H1 and H2 peptides stored in βig-h3 have been confirmed not to affect βig-h3-mediated cell adhesion, suggesting that certain amino acid required for cell adhesion locates not in H1 and H2 but in other sites in βig-h3. In order to support the above, the homology between repeated fas-1 domain of βig-h3 and fas-1 domains of other proteins was analyzed by computer, resulting in the confirmation of the fact that there were many other preservative amino acids except H1 and H2 in βig-h3 that participated in cell adhesion.

Therefore, the present inventors tried to find out a preservative motif participating in cell adhesion and detachment activity, and to prepare a peptide containing thereof. As a result, the present inventors have prepared peptides NKDIL, EPDIM and their derivatives mediating cell adhesion and detachment by working with a 3β 1 integrin using the second and the forth domains of βig-h3 which is known as a cell adhesion molecule and have disclosed that two very preservative amino acids, aspartic acid (Asp) and isoleucine (Ile) which are located near H2 region in the second and the forth domains of βig-h3, are required amino acids for cell adhesion and detachment activity, leading to the application for a patent (Korea Patent Application #2000-25665).

As of today, there was no report that βig-h3 directly relates to diseases but βig-h3 seems to be related with some human cancers. The relation of βig-h3 expression with the progress of renal diseases, hepatic diseases, rheumatoid arthritis and cardiovascular diseases has not been explained yet and the possibility to take advantage of βig-h3 protein for a diagnosis of the diseases by measuring the amount of βig-h3 protein in body fluids has not been reported either.

Thus, the present inventors developed a method to measure the amount of βig-h3 using the recombinant protein prepared by linking many βig-h3 or the forth fas-1 domain of βig-h3 together as a standard protein and a diagnostic kit using the same. The present inventors completed this invention by confirming that the method and the kit of the present invention can be effectively used as sensitive diagnostic method for the extent of damage or progress of the renal diseases, hepatic diseases, rheumatoid arthritis or cardiovascular diseases.

### SUMMARY OF THE INVENTION

It is an object of the present invention to provide a method to measure the amount of βig-h3 protein using the βig-h3 protein or recombinant proteins including fas-1 domains of βig-h3 and a diagnostic kit using the same.

### BRIEF DESCRIPTION OF THE DRAWINGS

FIG. 1 is a diagram showing the structure of βig-h3 recombinant protein,
I, II, III and IV: each domain,
[symbol] and [symbol] : base sequence preservative area
A; βig-h3, B; human βig-h3, C; mouse βig-h3

FIG. 2 is a diagram showing the geometrical structure of βig-h3 D-IV recombinant proteins prepared by repeating βig-h3 IV domains,
A; βig-h3, B; βig-h3 D-IV(1×),
C; βig-h3 D-IV(2×),
D; βig-h3 D-IV(3×), E; βig-h3 D-IV(4×)

FIG. 3 is an electrophoresis photograph of separated βig-h3 recombinant protein,
1; human βig-h3, 2; mouse βig-h3

FIG. 4 is an electrophoresis photograph of βig-h3 D-IV (1×, 2×, 3×, 4×) proteins,
1; βig-h3 D-IV(1×), 2; βig-h3 D-IV(2×),
3; βig-h3 D-IV(3×), 4; βig-h3 D-IV(4×)

FIG. 5 is a photograph showing the result of Western blot using primary antibody, by which human βig-h3 and mouse βig-h3 were confirmed, 1; human βig-h3, 2; mouse βig-h3

FIG. 6 is a diagram showing the principle of enzyme-linked immunosorbent assay (ELISA),

FIG. 7 is a graph showing the quantitative ratios of the primary antibody,
♦; 1:200, ▪; 1:400, ▴; 1:800,
x; 1:1600, ※; 1:2000, ; 1:3200

FIG. 8 is a graph showing the quantitative ratios of the secondary antibody,
A; fixed primary antibody at 1:1600,
B; fixed primary antibody at 1:2000,
♦; diluted secondary antibody at 1:1000,
▪; diluted secondary antibody at 1:2000,
; diluted secondary antibody at 1:3000

FIG. 9 is a graph showing the coating concentration of human βig-h3 protein,
♦; 0.5 μg/ml, ▪; 1.0 μg/ml

FIG. 10 is a graph showing that both human βig-h3 protein and mouse βig-h3 protein can be used as standard proteins, which was confirmed by cross-test,
♦; human βig-h3 protein coating concentration 0.5 μg/ml, primary anti-human βig-h3 antibody 1:2000, secondary antibody 1:2000,
▪; human βig-h3 protein coating concentration 0.5 μg/ml, primary anti-mouse βig-h3 antibody 1:2000, secondary antibody 1:2000,
▴; mouse βig-h3 protein coating concentration 0.5 μg/ml, primary anti-human βig-h3 antibody 1:2000, secondary antibody 1:2000,
x; mouse βig-h3 protein coating concentration 0.5 μg/ml, primary anti-mouse βig-h3 antibody 1:2000, secondary antibody 1:2000

FIG. 11 is a graph showing that recombinant βig-h3 D-IV(1×) protein and recombinant βig-h3 D-IV(4×) protein can be used as standard proteins, which was confirmed by cross-test,
♦ of A; βig-h3 D-IV(1×) coating concentration 0.5 μg/ml, primary anti-human βig-h3 antibody 1:2000, secondary antibody 1:2000,
▪ of A; βig-h3 D-IV(4×) coating concentration 0.5 μg/ml, primary anti-human § ig-h3 antibody 1:2000, secondary antibody 1:2000,
♦ of B; βig-h3 D-IV(1×) coating concentration 0.5 μg/ml, primary anti-mouse a ig-h3 antibody 1:2000, secondary antibody 1:2000,
▪ of B; βig-h3 D-IV(4×) coating concentration 0.5 μg/ml, primary anti-mouse μ ig-h3 antibody 1:2000, secondary antibody 1:2000

FIG. 12 is a photograph of an immunohistochemical-staining showing the expression pattern of βig-h3 in renal tissue,
[symbol] of A; expression pattern at basal membrane of S3 proximal tubular cell,

[symbol] of B; expression pattern at basal membrane of Bowman's capsule of glomerulus,
→ of B; expression pattern at basal membrane of cortical thick ascending limb cell

FIG. 13 is a graph showing the levels of βig-h3 in urine of diabetes-induced rats,
▪; control group,
□; diabetes-induced rats by treatment of streptozotocin

FIG. 14 is a graph showing the individual level of βig-h3 in urine of diabetes-induced rats of FIG. 13,

FIG. 15 is a graph showing the level of βig-h3 in urine obtained from each a normal rat, a rat with nephron underdose, a rat with chronic rejection, a rat with recurrent GN and a rat showed CyA toxicity,

FIG. 16 is a graph showing the different concentrations of βig-h3 protein by the day that were measured with urine samples of patients who have been under the treatment of plasmapheresis since focal segmental glomerulosclerosis (FSGS) was re-developed after kidney transplantation,

FIG. 17 is a graph showing the concentrations of βig-h3 protein in urine taken from a living donor, cadaver donor, a patient with underdose and rejection that were measured before and after kidney transplantation,

FIG. 18 is a photograph of an immunohistochemical-staining showing the expression pattern of βig-h3 protein in the injured blood vessels of diabetes-induced mouse,
A; normal blood vessels,
B; injured blood vessels, L; lumen

FIG. 19 is a graph showing the expression pattern of βig-h3 protein in the culture of vascular smooth muscle cells,
*; p<0.05, **; p<0.01

### DETAILED DESCRIPTION OF THE INVENTION

To achieve the above object, the present invention provides a method for measuring the amount of βig-h3 protein.

The present invention also provides a diagnostic kit for the renal diseases, hepatic diseases, rheumatoid arthritis or cardiovascular diseases using the same.

Further features of the present invention will appear hereinafter.

The method for measuring the amount of βig-h3 of the present invention consists of following steps:
1) Preparing βig-h3 protein or recombinant proteins containing βig-h3 fas-1 domain, their fragments or derivatives;
2) Preparing specific ligands against the above recombinant proteins, their fragments or derivatives of the above step 1; and
3) Measuring the amount of βig-h3 protein of samples with the method using binding reaction of ligands of the above step 2 with the recombinant proteins, their fragments or derivatives of the above step 1.

In the step 1, βig-h3 protein is either a human βig-h3 protein having amino acid sequence represented by SEQ. ID. No 3 or a mouse βig-h3 protein having amino acid sequence represented by SEQ. ID. No 5. The structural elements of human and mouse βig-h3 proteins are shown in FIG. 1. Hatched region and cross-hatched region of FIG. 1 show very well preserved sequences of repeated fas-1 domain I, II, III and IV and blank region represents RGD motif.

βig-h3 protein has 4 fas-1 domains. For the βig-h3 fas-1 domain of the above step 1, it is preferable to select one or more than two out of the first through the 4^{th} fas-1 domain of βig-h3 protein and is more preferable to use the 4^{th} fas-1 domain. The 4^{th} fas-1 domain could be used either individually or as a recombinant protein in which many fas-1 domains are repeatedly linked. For the recombinant protein, 1 to 10 fas-1 domains are required to be combined and using 1 to 4 fas-1 domains is more preferred. In the preferred embodiments of the present invention, the present inventors provided examples of using the 4^{th} fas-1 domain only and recombinant proteins prepared by linking two, three and 4 forth fas-1 domains of βig-h3 respectively.

The present inventors prepared proteins each represented by SEQ. ID. No 7, No 8, No 9 and No 10 having one of the 4^{th} fas-1 domains containing 502^{nd}-632^{nd} amino acids of βig-h3, two, three and four of those respectively and named them “βig-h3 D-IV(1×)”, “βig-h3 D-IV(2×)”, “βig-h3 D-IV(3×)” and “βig-h3 D-IV(4×)” (see FIG. 4).

Epitope of βig-h3 protein at which specific binding reaction with ligand is occurring and any other part of the protein containing peptides hydrolyzed by protease can be used as fragments of the recombinant protein. Derivatives of the recombinant protein of the present invention can be prepared by covalent bond including phosphorylation or glycosylation, and non-covalent bond including ionic bond, coordinate bond, hydrogen bond, hydrophobic bond or van der Waals' bond. If fragments of the derivatives of the above recombinant proteins could be specifically bound to ligands, they would be included in the category of the proteins of the present invention.

For the preparation of the standard protein of the present invention, the construction of expression vector and the transformation could be performed by the conventional method.

In the step 2, ligands that are specifically binding to βig-h3, βig-h3 fas-1 domain, fragments or derivatives thereof can be confirmed by observing the binding reaction of ligands with the protein or recombinant protein of the step 1. There are many kinds of ligands such as antibody, RNA, DNA, organic compounds including lipid, protein or organic salts, or inorganic compounds including metal ions or inorganic salts, and preferable ligand is a primary antibody against βig-h3 or βig-h3 fas-1 domain of the step 2 made by using the protein or the recombinant protein (fragments or derivatives included) of the step 1 as an antigen. The primary antibody can be prepared by the conventional method and monoclonal antibody or polyclonal antibody can be used.

In the step 3, the amount of βig-h3 protein included in sample was measured using the specific binding reaction of ligand with βig-h3 protein, its fragments or derivatives. Where ligand-binding reaction is occurring, even pieces of those fragments or derivatives can be used. Quantification assay using antigen-antibody binding reaction in which βig-h3 protein is used as an antigen is preferably used. It is more preferable to select one way from a group consisting of immunoblotting (Current Protocols in Molecular Biology, vol 2, chapter 10.8; David et al., Cells (a Laboratory manual), vol 1, chapter 73), immunoprecipitation (Current Protocols in Molecular Biology, vol 2, chapter 10.16; Cells(a Laboratory manual), vol 1, chapter 72), ELISA (Current Protocols in Molecular Biology, vol 2, chapter 11.2; ELISA Theory and Practice, John R. Crowther; The ELISA Guidebook, John R. Crowther), RIA (Radioimmuno assay) (Nuklearmedizin 1986 August; 25 (4): 125-127, Tumor markers as target substances in the radioimmunologic detection of malignancies. von Kleist S; Mariani G. Ann Oncol 1999; 10 Suppl 4: 37-40), protein chip (Daniel Figeys et. al, Electrophoresis 2001, 22, 208-216; Albala J S. Expert Rev Mol Diagn 2001 July; 1 (2): 145-152), rapid assay (Kasahara Y and Ashihara Y, Clinica Chimica Acta 267 (1997), 87-102; Korea Patent Application #2000-46639) or microarray (Vivian G. cheung et al, Nature genetics 1999, 21, 15-19; Robert J. Lipshutz et al, Nature genetics 1999, 21, 20-24; Christine Debouck and Peter N. Goodfellow, Nature genetics 1999, 21, 48-50; DNA Microarrays, M. Schena), and ELISA is the most preferable method. Mass-analysis of samples is also possible using biological microchip and automatic microarray system along with ELISA, and simple self-diagnostic method using urine can be developed therefrom.

According to the preferable embodiments of the present invention, the method for measuring the amount of βig-h3 protein with competition assay using ELISA comprises the following steps;
1) Coating βig-h3 protein or recombinant protein containing βig-h3 fas-1 domain, its fragments or derivatives to matrix;
2) Reacting antibody against the protein of the above step 1, its fragments or derivatives with sample;
3) Adding the reactant of the above step 2 to the coated protein of step 1 and waiting for reaction, and then washing thereof; and
4) Adding the secondary antibody to the reactant of the above step 3 for further reaction, and then measuring OD.

All kinds of matrix commonly used are good for the matrix of the above step i and especially, nitrocellulose membrane, polyvinyl plate (for example; 96 well plate), polystyrene plate and glass slide can be used as a matrix.

The secondary antibody of the above step 4 is labeled with coloring enzymes, fluorescent materials, luminous materials, radioisotopes or metal chelates. Every commonly used labeling materials are available for this invention and peroxidase, alkaline phosphatase, β-D-galactosidase, malate dehydrogenase, staphylococcus nuclease, horseradish peroxidase, catalse and acetylcholine esterase are preferable coloring enzymes. As for fluorescent materials, fluorescein isothiochanate, phycobilin protein, rhodamine, phycoerythrin, phycocyanin, orthophthalic aldehyde, etc are preferably used.

As another labeling materials for the secondary antibody in addition to coloring enzymes or fluorescent materials, luminous materials such as isoluminol, lucigenin, luminol, acridiniumester, imidasol, acridine salt, luciferin, luciferase and aequorin or radioisotopes such as ¹²⁵I, ¹²⁷I, ¹³¹I, ¹⁴C, ³H, ³²P and ³⁵S are preferably used. Besides, micromolecular heptenes like biotine, dinitrophenyl, pyridoxil or fluoresamine can be also conjugated with antibody.

In the case of using coloring enzymes in step 4, coloring substrates should be used to measure the activity of the enzyme and every material that are able to develop color of the enzyme bound to the secondary antibody can be used as a coloring substrate. 4-chloro-1-naphtol (4CN), Diaminobenzidine (DAB), Aminoethyl carbazole (AEC), 2,2′-Azino-bis(3-ethylbenzothiazoline-6-sulfonic acid) (ABTS), o-Phenylenediamine (OPD) and Tetramethyl Benzidine (TMB) are preferably used as coloring substrates.

As for the samples of the above step 2, all kinds of body fluids of patients suffering from βig-h3 related diseases can be used. Especially, urines, bloods or synovial fluids of patients suffering from renal diseases, hepatic diseases, rheumatoid arthritis or cardiovascular diseases are preferable.

In order to confirm whether the method for measuring the amount of βig-h3 protein of the present invention is correct, the present inventors used recombinant protein containing mouse βig-h3 or the 4^{th} fas-1 domain of βig-h3 as a standard protein and compared the result with that from using human βig-h3 as a standard protein.

The optimum coating concentration of human βig-h3 protein and the quantitative ratio of antibody were determined for the method for measuring βig-h3 of the present invention. The best quantitative ratio of the primary anti-human βig-h3 antibody was 1:1600 and 1:2000 (see FIG. 7), and the best quantitative ratio of the secondary antibody was 1:2000 (see FIG. 8). The proper concentration of human βig-h3 protein was 1.0 μg/ml and 0.5 μg/ml, but 0.5 μg/ml was more preferable as coating concentration (see FIG. 9).

Therefore, the present inventors decided the optimum coating concentration of human βig-h3 standard protein to be 0.5 μg/ml and the best diluting ratio of the primary anti-human βig-h3 antibody and the secondary antibody to be 1:2000, respectively.

The present inventors also determined protein concentration and the quantitative ratio of the primary antibody and the secondary antibody using mouse βig-h3, recombinant βig-h3 D-IV(1×), ig-h3 D-IV(2×), ig-h3 D-IV(3×) and βig-h3 D-IV(4×). Precisely, made coating concentration of each protein at 0.5 μg/ml, diluted the primary anti-human βig-h3 antibody and the secondary antibody at 1:2000 respectively and performed quantitative assay. Diluted the primary anti-mouse βig-h3 antibody and the secondary antibody at 1:2000, and performed quantitative assay as well.

As a result, graphs with straight line were made for all the cases, suggesting the ratios were the best and the measuring range of them was between 11 ng/ml-900 ng/ml, meaning there was not much difference in the measuring range among them all (see FIG. 11 and FIG. 12).

From the above results, it was confirmed that standard protein could be any of human βig-h3, mouse D ig-h3, recombinant βig-h3 D-IV(1×), ig-h3 D-IV(2×), ig-h3 D-IV(3×) and βig-h3 D-IV(4×), and either anti-human βig-h3 antibody or anti-mouse βig-h3 antibody could be used as the primary antibody.

In this invention, the preferable coating concentration of standard protein is 0.1-2.0 μg/ml and 0.5-1.0 μg/ml is more preferable. The preferable diluting ratio of the primary and the secondary antibody is 1:400-1:3200 and 1:2000 is more preferable.

The present invention provides a diagnostic kit for renal diseases, hepatic diseases, rheumatoid arthritis or cardiovascular diseases, with which the diseases are diagnosed by measuring the amount of βig-h3 protein in the body fluids of patients.

The diagnostic kit of the present invention includes βig-h3 protein or recombinant proteins of fas-1 domain in the βig-h3 protein (including their fragments or their derivatives) and their ligands. At this time, as preferable specific ligands, antibodies against βig-h3 protein or βig-h3 fas-1 domains are used. The kit can additionally include buffer solution, secondary antibody, washing solution or coloring substrate.

The diagnostic kit of the present invention is available for the diagnosis of various diseases such as renal diseases, hepatic diseases, rheumatoid arthritis or cardiovascular diseases by measuring the amount of βig-h3 protein in the body fluids.

It is possible to diagnose renal diseases by measuring the amount of βig-h3 protein on the basis of the fact that βig-h3 expression is induced by TGF-β that plays an important role in the development of renal diseases. For the confirmation of the above, measured the amount of βig-h3 in urine of diabetic patients. As a result, the amount of βig-h3 in urine of patients with diabetic renal diseases including microalbuminuria was about five-fold higher than that of normal person. Some diabetic patients without renal diseases also showed higher βig-h3 amount than normal. Considering the above result, βig-h3 level in urine seems to reflect the extent of renal damage and high βig-h3 level of some diabetic patients without renal diseases suggests that their kidneys are already damaged to some degree, though not showing any clinical troubles yet. Therefore, measuring the amount of βig-h3 in patients' urine is a highly sensitive and important diagnostic method that can reflect the damage of kidneys in the early stage.

In order to confirm whether the βig-h3 concentration in a diabetic patient's urine can reflect the damage of a kidney in the early stage, measured the βig-h3 concentration of a diabetic animal. As a result, the βig-h3 concentration was 4-fold increased 5 days after inducing diabetes (see FIG. 13). Observed the changes of βig-h3 concentration in each individual after inducing diabetes, resulting in the great increase of βig-h3 concentration in urine after inducing diabetes (see FIG. 14). On the 5^{th} day after inducing diabetes, blood urea and creatine were normal and kidney tissues seemed normal. Thus, the great increase of βig-h3 amount in urine on the fifth day suggests that there was the minimum damage in kidney already, which could not be detected by the traditional test methods.

The present inventors further confirmed the relation between kidney damage and βig-h3 concentration by measuring βig-h3 amount in urine of preoperative and postoperative patients with kidney transplantation. As a result, the high βig-h3 concentration of a preoperative patient dropped gradually after successful operation. But in the case of No. 5 patient whose kidney function was not recovered even after operation, the βig-h3 concentration was still great (see FIG. 2). Considering all the above results, it is for sure that the βig-h3 concentration sensitively reflects the extent of kidney damage.

The present inventors also measured the βig-h3 concentration in urine of renal failure patients. As a result, all of those renal failure patients showed great βig-h3 concentration in their urine. Thus, it was confirmed again that βig-h3 amount in urine reflects kidney damage sensitively even in the early stage, so that measuring the βig-h3 amount is very important diagnostic method for various renal diseases (see Table 3).

Determining if a chronic hepatitis patient is developing to a hepatocirrhosis patient is very important but there is no way to catch that so far. The most crucial factor for the development of hepatocirrhosis is TGF-β. Thus, βig-h3 whose expression is induced by TGF-β could be possibly increased in blood as hepatocirrhosis goes on. If so, the amount of βig-h3 can also reflect the extent of hepatocirrhosis. In fact, βig-h3 expression was confirmed to be greater as hepatocirrhosis became serious by immunohistological test with liver tissues of hepatitis patients. The present inventors subdivided patient's condition into several grades and stages based on the biopsy results of chronic hepatitis patients and investigated blood βig-h3 concentration of each stage and grade. Chronic hepatitis patients showed higher blood βig-h3 concentration than normal people. βig-h3 concentration of lower stage and grade was confirmed to be higher than that of higher stage and grade (see Table 5). Condition of a patient in grade 3 and stage 3 is that hepatocirrhosis has been developed seriously and its activity went through the peak already. Meanwhile, a patient in grade 1 and 2 and stage land 2 shows the condition that inflammatory reaction is developing very actively. Thus, βig-h3 concentration implies the activity of hepatocirrhosis, so that the development of hepatocirrhosis can be observed by measuring blood βig-h3 concentration regularly.

βig-h3 concentration in synovial fluid of rheumatoid arthritis patients and osteoarthritis patients was also measured. As a result, two-fold higher βig-h3 concentration in synovial fluid of rheumatoid arthritis patients was observed, suggesting that measuring βig-h3 concentration in synovial fluid can be an effective way to diagnose osteoarthritis and rheumatoid arthritis (see Table 6).

In addition, the expression patterns of βig-h3 in normal and damaged blood vessels of diabetic mice were investigated by immunohistochemical methods in order to confirm the relation between the expression of βig-h3 and vascular diseases. As a result, βig-h3 protein was expressed much greatly in damaged blood vessels of diabetic mice than in normal blood vessels (see FIG. 18). Based on that βig-h3 expression is induced by TGF-β that plays an important role in the development of vascular diseases, TGF-β1 inducing βig-h3 expression in vascular smooth muscle cells forming blood vessels was investigated. As a result, it was confirmed that βig-h3 expression increases as the amount of TGF-β1 increases (see FIG. 19).

The expression of βig-h3 in blood and tissues reflects the damage of them. Thus, it was confirmed that the method for measuring the amount of βig-h3 protein of the present invention can be effectively used for the diagnosis of various vascular diseases.

Therefore, the diagnostic kit measuring the amount of βig-h3 protein of the present invention is very effective in use since it reflects the extent of damage and progress of renal diseases, hepatic diseases, rheumatoid arthritis or cardiovascular diseases.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Preparation of Standard Proteins and Primary Antibodies

<1-1> Separation of Human βig-h3 and Mouse βig-h3

The present inventors have prepared human and mouse βig-h3 proteins. The structural elements of human and mouse βig-h3 proteins are shown in FIG. 1. Hatched region and cross-hatched region of FIG. 1 show very well preserved sequences of repeated fas-1 domain I, II, III and IV and blank region represents RGD motif. βig-h3 cDNA (pBS βig-h3; obtained by cloning cDNA of human skin papilloma cells) having a base sequence represented by SEQ. ID. No 2 cloned in pBluescript SK (−) vector was digested with Nde I and Bgl II, resulting in the preparation of DNA fragments having blunt ends. The above DNA fragments were subcloned into EcoR V and EcoR I sites of pET-29β vector (purchased from Novagen). The protein having a amino acid sequence of 69-653 amino acids of βig-h3 represented by SEQ. ID. No 3 was separated and named human βig-h3.

Next, βig-h3 cDNA was digested with BamH I and Xho I, resulting in the preparation of DNA fragments having a base sequence represented by SEQ. ID. No 4. The above DNA fragments were subcloned into BamH I and Xho I sites of pET-29β vector. The protein having a amino acid sequence of 23-641 amino acids of βig-h3 represented by SEQ. ID. No 5 was separated and named mouse βig-h3.

In order to express the above human and mouse βig-h3 proteins, E. coli BL21 (DE3) cells were transformed. The transformants were cultured in LB medium containing kanamicine (50 μg/ml) at 37° C. until their OD₅₉₅ was reached to 0.5-0.6. During the culture, the expression of βig-h3 protein was induced by treating 1 mM isopropyl-β-D-(−)thiogalactopyranoside (IPTG) at 37° C. for 3 hours.

Pellets of E. coli cells were resuspended in cell lysis buffer (50 mM Tris-HCl, pH 8.0, 100 mM NaCl, 1 mM EDTA, 1% Triton X-100, 1 mM phenylmethane sulfonyl fluoride (referred as “PMSF” hereinafter) and 0.5 mM DTT), and then crushed by ultrasonification. The procedure was repeated 5 times.

The above solution was centrifuged and the insoluble inclusion bodies containing βig-h3 were dissolved in 20 mM Tris-HCl buffer solution containing 0.5 M NaCl, 5 mM imidazol and 8 M urea. The proteins were purified by using Ni-NTA resin (Qiagen). The proteins were dialyzed one after another in 20 mM Tris-Cl buffer solution containing 50 mM NaCl with urea starting from high concentration to low concentration for the purification and the results were confirmed by SDS-PAGE.

As a result, it was confirmed that the human βig-h3 and the mouse βig-h3 proteins of the present invention were purified (FIG. 2).

<1-2> Construction and Separation of βig-h3 D-IV(1×) and βig-h3 D-IV(4×)

The DNA fragment represented by SEQ. ID. No 6 encoding the 4^{th} domain that corresponds to 498^{th}-637^{th} amino acids of human βig-h3 represented by SEQ. ID. No 1 was amplified by PCR. The PCR product was cloned into pET-29β vector to construct the expression vector of the 4^{th} domain. The present inventors named the expression vector of the 4^{th} domain “βig-h3 D-IV”.

Base sequence that corresponds to the 4^{th} domain was synthesized by PCR, and the 3′ end of the PCR product was blunted by using klenow fragment. This PCR product was inserted into EcoR V site of the above expression vector pβig-h3 D-IV, and named pβig-h3 D-IV(2×). Inserted fragment of pβig-h3 D-IV(2×) was digested with EcoR V and Xho I, and the 3′ end of the fragment was blunted by using klenow fragment. This fragment was inserted into EcoR V site of pβig-h3 D-IV, and named pβig-h3 D-IV(3×). The fragment having blunted 3′ end was also inserted into EcoR V site of pβig-h3 D-IV(2×), and named pβig-h3 D-IV(4×) (FIG. 3). His-tag was made by linking 6 histidine residues to carboxyl terminal of the DNA fragment to purify proteins with Ni-NTA resin (Qiagen).

E. coli BS21(DE3) cells were transformed with the expression vectors. The transformants were cultured in LB medium containing kanamicine (50 μg/ml). Pellets of E. coli cells were resuspended in cell lysis buffer (50 mM Tris-HCl, pH 8.0, 100 mM NaCl, 1 mM EDTA, 1% Triton X-100, 1 mM phenylmethane sulfonyl fluoride (referred as “PMSF” hereinafter) and 0.5 mM DTT), and then crushed by ultrasonification. The procedure was repeated 5 times. The above solution was centrifuged to obtain supernatants. The proteins were purified by using Ni-NTA resin (Qiagen) from the supernatants, and confirmed with SDS-PAGE.

As a result, it was confirmed that βig-h3 D-IV(1×) having an amino acid sequence represented by SEQ. ID. No 7, βig-h3 D-IV(2×) having an amino acid sequence represented by SEQ. ID. No 8, βig-h3 D-IV(3×) having an amino acid sequence represented by SEQ. ID. No 9 and βig-h3 D-IV(4×) having an amino acid sequence represented by SEQ. ID. No 10 proteins were expressed. All the above proteins contained the 4^{th} domain of human βig-h3 (FIG. 4).

<1-3> Preparation and Separation of Primary Antibody

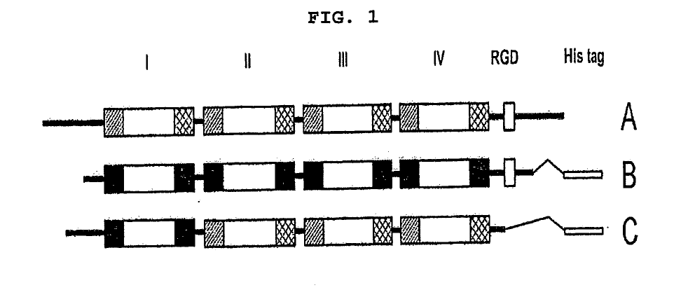

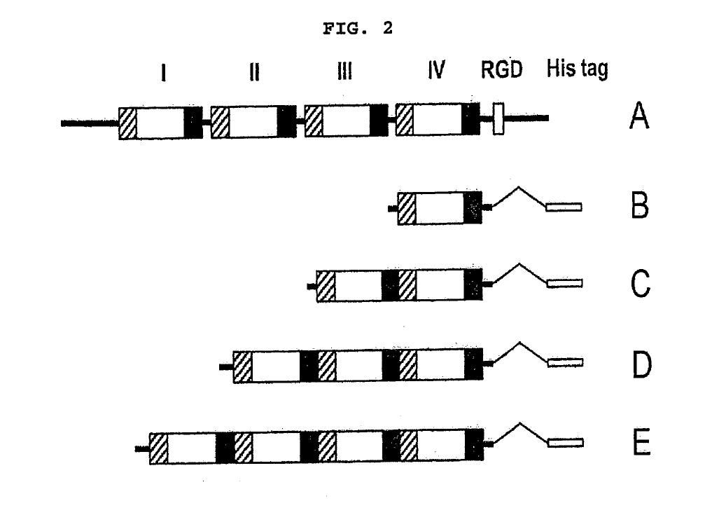

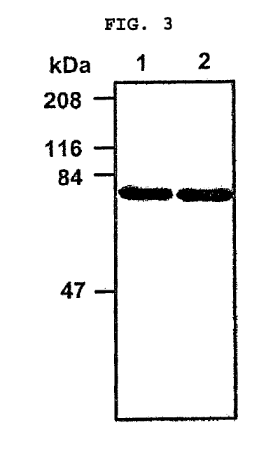

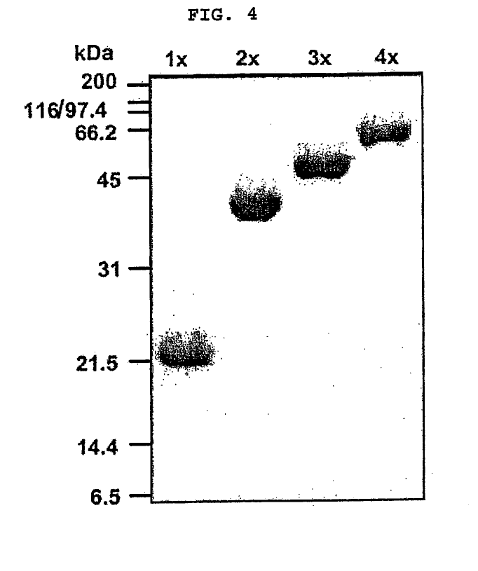

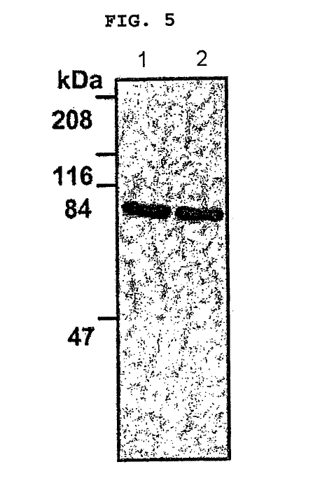

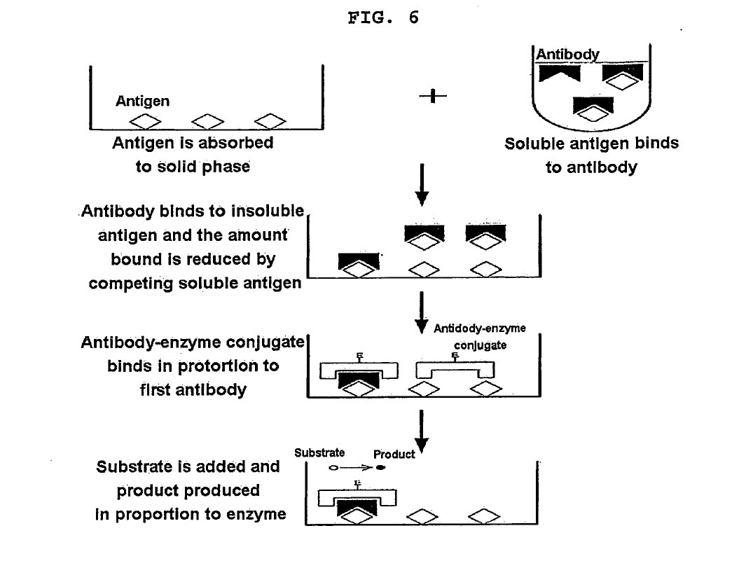

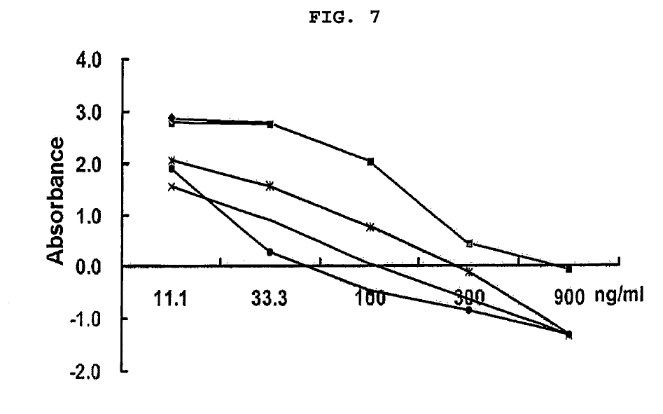

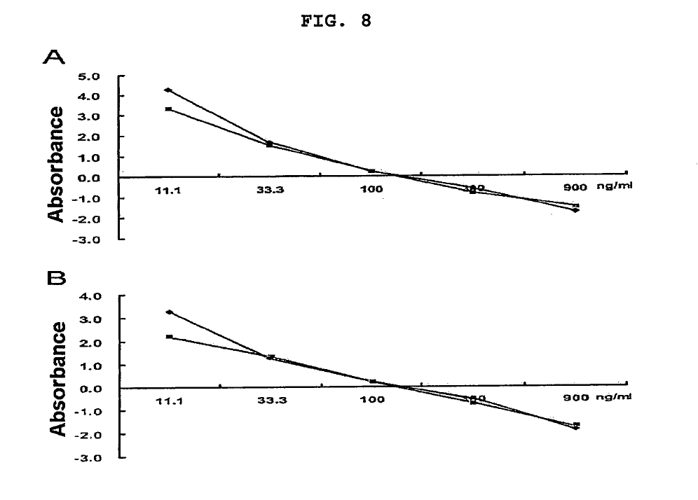

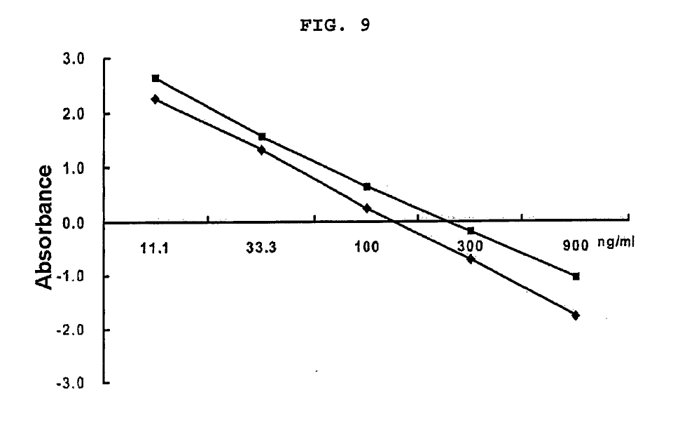

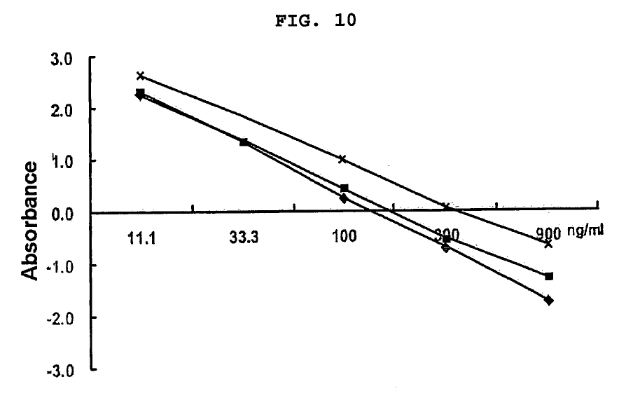

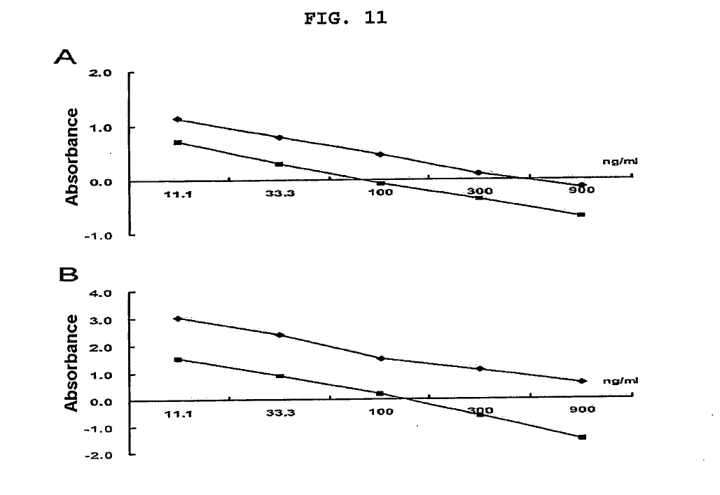

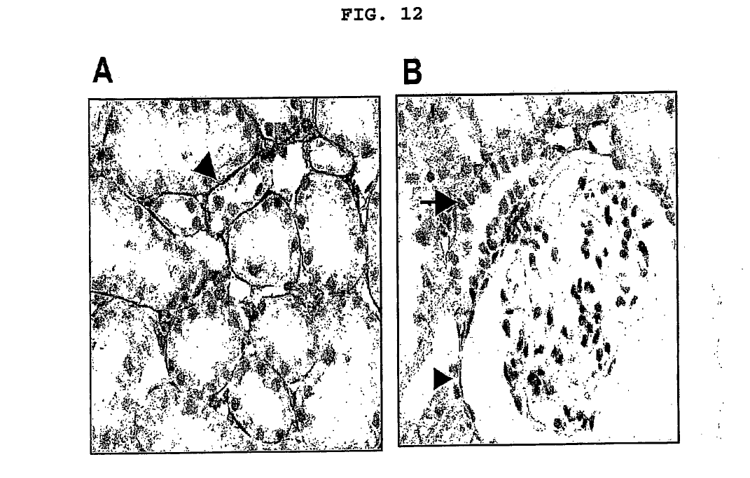

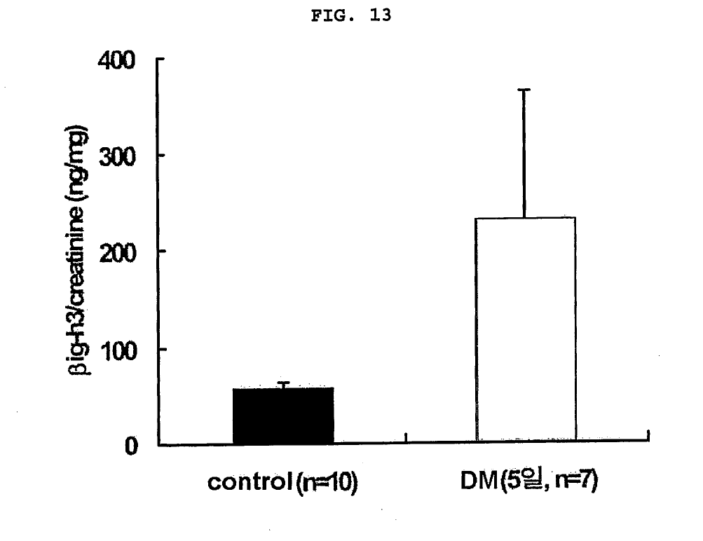

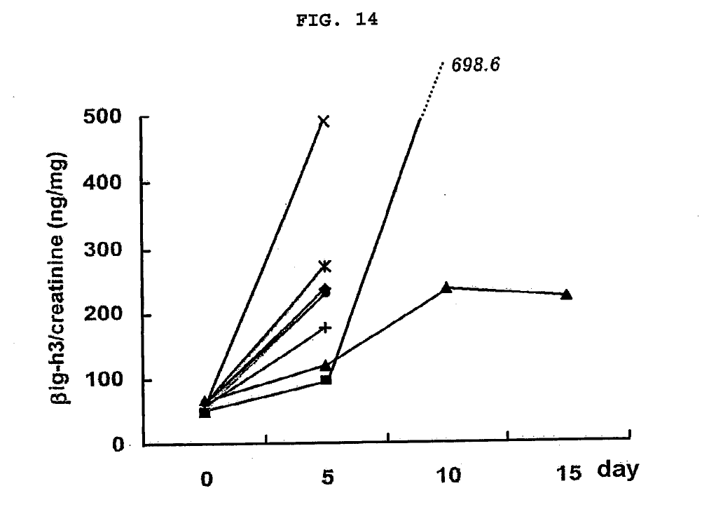

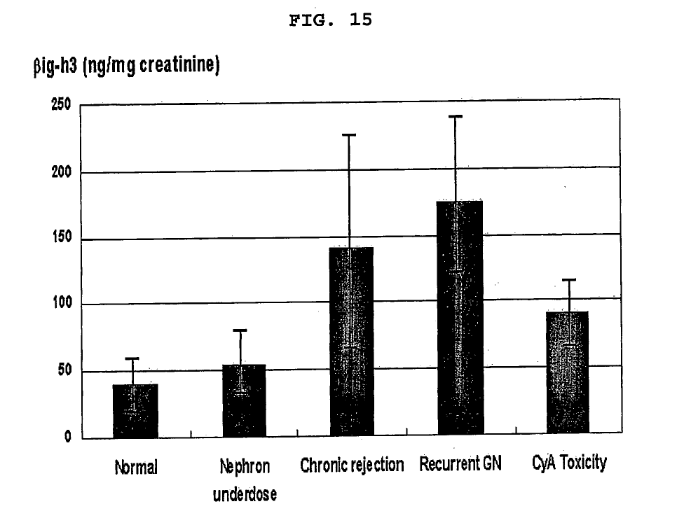

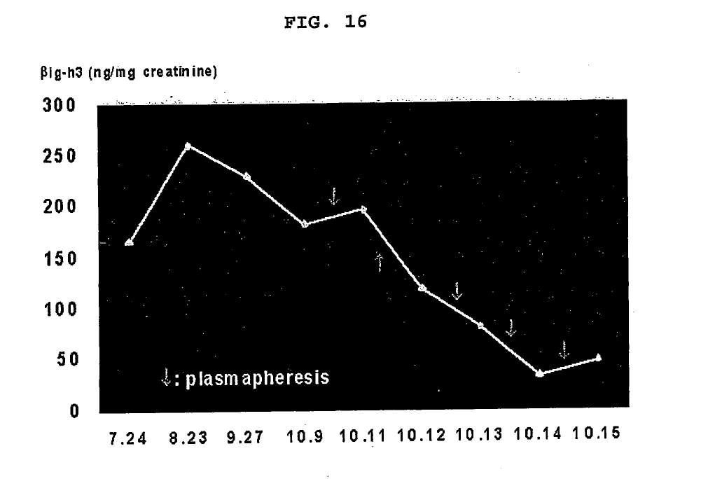

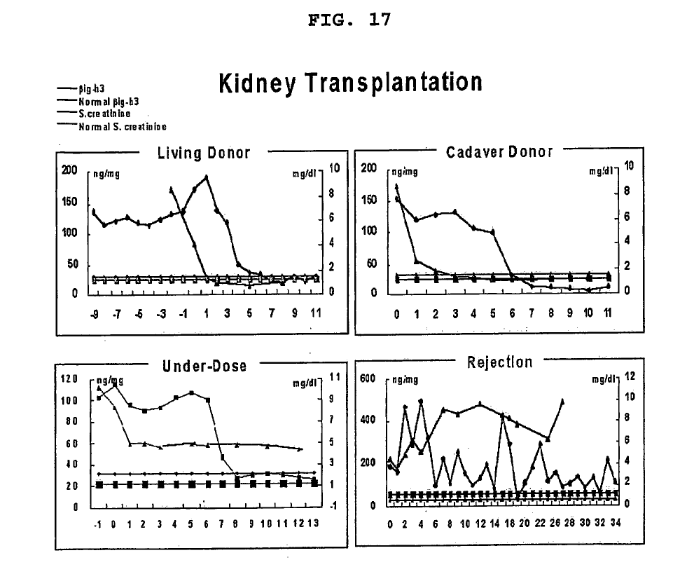

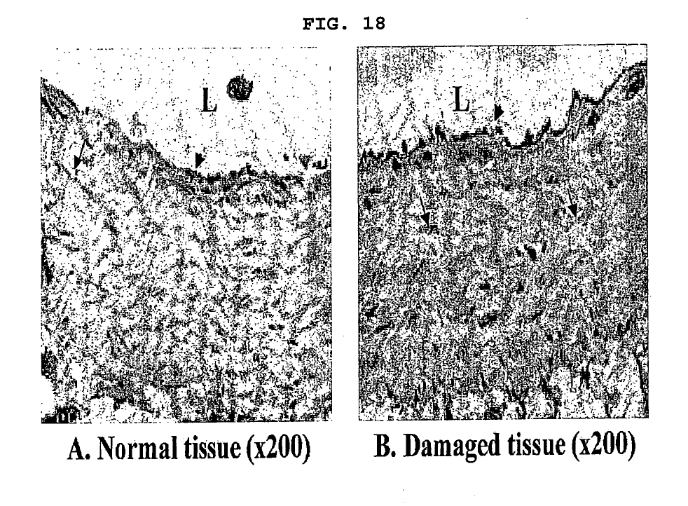

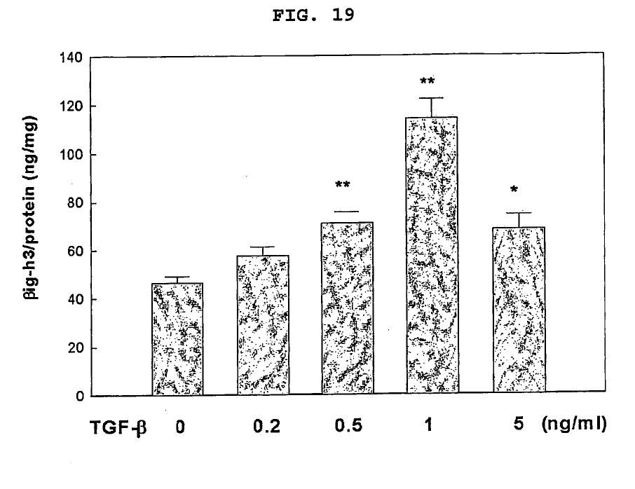

The primary antibody was prepared by using human βig-h3 and mouse βig-h3 proteins separated in Example <1-1> as an antigen. The proteins were subcutaneously injected on the back of rabbits. For the first injection, 200 μg of proteins were mixed with complete Freund's adjuvant and then injected. For the 2^{nd} to 5^{th} injection, 100 μg of proteins were mixed with incomplete Freund's adjuvant and then injected at 3-week intervals. Venous blood was collected and left at room temperature for 2 hours. Following centrifugation (10,000×g, 10 minutes), the supernatants containing the primary antibody were obtained. The supernatants were kept at −20° C. for further usage (FIG. 5).

### Example 2

### Determination of Coating Concentration of Human βig-h3 Protein and Quantitative Ratio of Antibody

<2-1> Determination of Quantitative Ratio of the Primary Antibody

In order to determine the quantitative ratio of the primary antibody to human βig-h3 protein, the human βig-h3 was diluted (0.5 μg/ml) with 20 mM carbonate-bicarbonate solution (pH 9.6, 0.02% sodium azide contained). The βig-h3 solution was added in each well of 96-well plate (200 μl/well) and coated thereof at 4° C. for overnight. The primary anti-human βig-h3 antibody was serially diluted with diluting solution (saline-phosphate buffer solution/Tween 80) at 1:200, 1:400, 1:800, 1:1600, 1:2000 and 1:3200, and added into the coated 96-well plate. The secondary antibody (1:5000) was also added thereto and reacted thereof at room temperature for 1 and half hours. Substrate solution (prepared by dissolving o-phenylendiamine in methanol (10 mg/ml), diluting with distilled water at 1:100, and mixing with 10 μl of 30% hydrogen peroxide solution) was also added thereto and reacted thereof at room temperature for 1 hour. The reaction was terminated by adding 50 μl of 8 N sulfuric acid solution, and ELISA was performed (O.D 492 nm).

As a result, it was confirmed that the best quantitative ratio of the primary anti-human βig-h3 antibody was 1:1600 and 1:2000 (FIG. 7).

<2-2> Determination of Quantitative Ratio of Secondary Antibody

In order to determine the quantitative ratio of the secondary antibody, the human βig-h3 protein was coated on the plate (0.5 μg/ml). Added the primary anti-human βig-h3 antibody thereto (1:1600 and 1:2000) Added the secondary antibody thereto (1:1000, 1:2000 and 1:3000 respectively) and reacted thereof. ELISA was performed with the same method as the above Example <2-1>.

As a result, it was confirmed that the best quantitative ratio of the secondary antibody was 1:2000 (FIG. 8).

<2-3> Determination of Coating Concentration of Human βig-h3 Protein

In order to determine the coating concentration of human βig-h3 protein, the primary anti-human βig-h3 antibody was diluted at 1:2000, the secondary antibody was diluted at 1:2000, the human βig-h3 protein was coated on the plate at 0.5 μg/ml and 1.0 μg/ml respectively, and then ELISA was performed.

As a result, it was confirmed that the proper concentration of human βig-h3 protein was both 1.0 μg/ml and 0.5 μg/ml, but 0.5 μg/ml was more preferable as coating concentration since R² value approaches 1 best with that concentration (FIG. 9).

From the above results, the present inventors decided the optimum coating concentration of human βig-h3 standard protein to be 0.5 μg/ml and the best diluting ratio of the primary anti-human βig-h3 antibody and the secondary antibody to be 1:2000, respectively.

The values obtained from the above result were log transformed by Robard formula (Robard, 1971) represented by the below <Mathematical Formula 1>. Resultingly, a line was formed from 11 ng/ml to 900 ng/ml, which was the possible range in measurement. It was also confirmed that measurement was possible even to the range of 10 ng/ml with the above reaction condition (FIG. 10).
log b=log e^{b/(100-b)} <Mathematical Formula 1>

In the above formula, b represents the percentage to OD of the well that does not include any antigen in each concentration.

### Example 3

### Measurement of Quantitative Range of Mouse βig-h3, Recombinant βig-h3 D-IV(1×) and 3 ig-h3 D-IV(4×) by Cross-Test

The present inventors also determined protein concentration and the quantitative ratio of the primary and the secondary antibody using mouse βig-h3, recombinant βig-h3 D-IV(1×) and βig-h3 D-IV(4×). Particularly, made coating concentration of each protein 0.5 μg/ml and the quantitative ratio of the primary anti-human βig-h3 antibody and the secondary antibody to be 1:2000 for the experiments. Regulated the quantitative ratio of the primary anti-mouse βig-h3 antibody and the secondary antibody to be 1:2000 as well.

As a result, graphs with straight line were made for all the cases, suggesting the ratio was the best and the ranges of them were between 11 μg/ml and 900 ng/ml, meaning there were not much differences in the range of measurement (FIG. 11 and FIG. 12).

From the above results, it was confirmed that standard protein could be any of human βig-h3, mouse βig-h3, recombinant βig-h3 D-IV(1×) and βig-h3 D-IV(4×), and either anti-human βig-h3 antibody or anti-mouse βig-h3 antibody could be used as the primary antibody.

### Example 4

### Relationship Between Renal Diseases and βig-h3 Expression

<4-1> Measurement of βig-h3 in Diabetics

The present inventors have confirmed the relationship between renal diseases and βig-h3 expression on the basis of the fact that βig-h3 expression is induced by TGF-β that plays an important role in the development of renal diseases. For the confirmation, measured the amount of βig-h3 in urine of diabetics. Particularly, mixed 110 μl of urine of diabetic and 110 μl of the primary antibody (1:1000) in a round-bottomed plate, and cultured thereof at 37° C. for 1 hour. Added 200 μl of the above mixture to βig-h3-coated plate and reacted thereof at room temperature for 30 minutes. Stopped the reaction by adding secondary antibody-substrate stop solution, and performed ELISA (O.D 492 nm.

TABLE 1
Concentration of β ig-h3 in diabetics' urine

| Samples | β ig-h3 (ng/ml) |
|---|---|
| Normal | 31.0 (n = 93, ±8.6) |
| Type II DM | 101.9 (n = 51, ±17.1) |
| Type II DM + microalbuminuria | 127.4 (n = 30, ±27.7) |
| Type II DM + overt | 105.4 (n = 19, ±14.9) |
| proteinuria | |
| Type II DM + CRF | 153.6 (n = 93, ±28.1) |

As a result, the amount of βig-h3 in urine of diabetic renal disease patients including microalbuminuria was about five-fold higher than that of normal. Some diabetic patients without renal diseases also showed higher βig-h3 amount than normal. Considering the above results, βig-h3 level in urine seems to reflect the extent of renal damage and high βig-h3 level of some diabetic patients without renal diseases suggests that their kidneys have already been damaged to some degree, though not showing any clinical troubles yet. Therefore, measuring the amount of βig-h3 in patients' urine is a highly sensitive and important diagnostic-method that can reflect the damage of kidneys in the early stage.

<4-2> Measurement of βig-h3 in Diabetic Animal Model

In order to confirm whether the βig-h3 concentration in diabetic's urine can reflect the renal damage in the early stage, the present inventors measured the βig-h3 amount of diabetic animals.

Diabetes was induced in Sprague-Dawley (SD) rats by injecting streptozotosin (60 mg/kg), a kind of diabetes-inducing drug, into the peritoneal cavity of the rats. Confirmed that diabetes was induced by measuring the blood-glucose of the rats. Taken urines from the rats on the fifth day after inducing diabetes, and measured the βig-h3 amount with the same method of Example <4-1>.

As a result, the βig-h3 amount was 4-fold increased 5 days after inducing diabetes (56.9±6.4 ng/creatine mg:230.4±131.8 ng/creatine mg, FIG. 13). Observed the change of βig-h3 amount in each individual after inducing diabetes, resulting in the great increase of βig-h3 amount in urine after inducing diabetes (FIG. 14). On the fifth day after inducing diabetes, blood urea and creatine were normal and renal tissues seemed normal. Thus, the great increase of βig-h3 amount in urine on the fifth day after inducing diabetes suggested that there was the minimum damage in kidney already, which could not be detected by the conventional methods.

<4-3> Measurement of βig-h3 in Patients Operated on Kidney Transplantation

The present inventors confirmed the correlation between renal damage and βig-h3 amount by measuring βig-h3 amount in urines of patients before and after kidney transplantation. The results were presented in Table 2.

TABLE 2
Changes of β ig-h3 concentration in patients before and after kidney transplantation

| Day/ | | | | | | | | | | | | | | Success |
|---|---|---|---|---|---|---|---|---|---|---|---|---|---|---|
| Patients | −6 | −5 | −4 | −3 | −2 | −1 | 0 | 1 | 2 | 3 | 4 | 5 | 6 | or not |
| 1 | | | | 376.9 | 199.2 | 105.6 | 59.1 | 67.6 | 84.5 | 63.1 | 61.2 | 39.7 | 9.9 | ◯ |
| 2 | | 149.2 | 147.3 | 133.5 | 159.5 | 148.3 | 147.3 | 96.0 | 74.0 | 40.7 | 20.3 | 27.9 | 26.4 | ◯ |
| 3 | 107.8 | 95.8 | 101.4 | 102.3 | 102.2 | 106.1 | 106.6 | 125.5 | 83.5 | 49.4 | 36.5 | 33.3 | 23.2 | ◯ |
| 4 | | | | | | | 298.8 | 208.1 | 140.5 | 169.9 | 188.4 | 76.3 | 24.4 | ◯ |
| 5 | | | | | | | 188.6 | 160.7 | 469.3 | 290.9 | 494.7 | 324.4 | — | X |

As a result, the high βig-h3 amount of pre-operative patients dropped gradually after successful operation. But in the case of No 5 patient whose renal function was not recovered even after kidney transplantation, the βig-h3 amount was still great. Considering all the above results, it is for sure that the amount of βig-h3 sensitively reflects the extent of kidney damage.

<4-4> Measurement of βig-h3 in Patients with Renal Failure

The present inventors measured the βig-h3 amount in urines of patients with renal failure. As a result, all of those patients showed great βig-h3 amount in their urines (Table 3).

TABLE 3
Concentrations of β ig-h3 in urines of patients with renal failure

| Samples | β ig-h3 (ng/mg) |
|---|---|
| Normal | 31.0 (n = 93, ±8.6) |
| Chronic renal | 335.4 (n = 9, ±56.0) |
| failure | |

4-5> Measurement of βig-h3 in Patients with Kidney Related Diseases

In order to investigate whether βig-h3 was differently expressed in patients with renal diseases, the present inventors measured the βig-h3 concentration in urines taken from patients who showed normal signs after kidney transplantation, patients whose transplanted kidney was smaller, patients who showed chronic rejection, patients with re-developed pyelitis and patients who had cyclosphorine toxicity with the same method of Example <4-1>.

As a result, patients with normal signs after kidney transplantation showed 39.4 ng/creatine mg of βig-h3 concentration at average while patients with chronic rejection, re-developed pyelitis and cyclosphorine toxicity showed greatly increased βig-h3 concentration (140.8, 175.4 and 90.9 ng/creatine mg, respectively) (FIG. 15, Table 4).

TABLE 4

| | Normal | Transplanted | | | |
|---|---|---|---|---|---|
| | after | with | | | |
| | kidney | small | Chronic | Pyelitis | Cyclosphorine |
| | transplantation | kidney | rejection | re-developed | toxicity |
| β ig-h3 | (n = 47) | (n = 16) | (n = 15) | (n = 6) | (n = 6) |
| Average | 39.4 ± 18.2 | 54.7 ± 23.0 | 140.8 ± 81.1 | 175.4 ± 65.8 | 90.9 ± 22.4 |
| Minimum | 9.4 | 17.9 | 48.8 | 83.2 | 64.6 |
| Maximum | 84.7 | 100.0 | 374.4 | 249.8 | 119.4 |

The present inventors also investigated if the increased βig-h3 concentration in patients with re-developed renal diseases was decreased again as treatment worked. As a result, urine βig-h3 concentration of patients who had blood plasma exchange to treat re-developed pyelitis after kidney transplantation was gradually decreased, suggesting urine βig-h3 concentration decreased while treatment was working. Thus, βig-h3 concentration could be used as a marker of treatment reaction (FIG. 16).

<4-6> Analysis of Effects of Kidney Transplantation on βig-h3 Concentration

In order to investigate the changes of urine βig-h3 concentration after kidney transplantation, the present inventors measured urine βig-h3 concentration of patients who had kidney transplantation everyday.

As a result, urine βig-h3 concentration of patients who had kidney transplantation successfully, regardless the kidney was given from a living person or a brain death person, was decreased gradually. Precisely, as for receiving kidney from a living person, urine βig-h3 concentration came back to normal level within 4-5 days after transplantation and as for receiving kidney from a brain death person, βig-h3 concentration came back to normal level within 6-7 days (FIG. 17).

Besides, urine βig-h3 concentration of patients who received small kidney came back to normal level after transplantation though their blood creatine values were still high, suggesting that the transplanted kidney worked normal although it could not filtrate waste products well enough because of its small size. Anyway, βig-h3 concentration reflecting the damage of kidney was back to normal (FIG. 17). Meanwhile, urine βig-h3 concentration of patients who had unsuccessful kidney transplantation fluctuated seriously.

Based on those results, urine βig-h3 concentration could be used as an effective marker for diagnosis of renal diseases in the early stages, for detecting progression of renal diseases and for determination of treatment effect since βig-h3 concentration reflects the damage of kidney well.

Resultingly, the present inventors confirmed that urine βig-h3 concentration reflects the damage of kidney in the early stages sensitively and is important and useful for diagnosis of various renal diseases.

### Example 5

### Relationship Between Hepatic Diseases and βig-h3 Expression

Determining if a chronic hepatitis patient is developing to a hepatocirrhosis patient is very important but there is no way to catch that so far. The most crucial factor for the development of hepatocirrhosis is TGF-β. Thus, βig-h3 whose expression is induced by TGF-could be possibly increased in blood as hepatocirrhosis goes on. If so, the amount of βig-h3 can also reflect the extent of hepatocirrhosis. In fact, βig-h3 expression was confirmed to be greater as hepatocirrhosis became serious by immunohistologic test with liver tissues of hepatitis patients. The present inventors subdivided patient's condition into several grades and stages based on the biopsy results of chronic hepatitis patients and investigated blood βig-h3 concentration of each stage and grade. Particularly, the present inventors collected blood from chronic hepatitis patients and measured the amount of βig-h3 with the same method of Example <4-1>. The results were presented in Table 5.

TABLE 5
Concentrations of β ig-h3 in blood of chronic hepatitis patients

| Grade | β ig-h3 (ng/mg) | Stage | β ig-h3 (ng/mg) |
|---|---|---|---|
| 0 | 146.2 | 0 | 146.2 |
| (Normal) | (n = 172, ±28.5) | (Normal) | (n = 172, ±28.5) |
| 1 | 196.6 | 1 | 193.4 |
| | (n = 16, ±30.6) | | (n = 20, ±30.2) |
| 2 | 190.0 | 2 | 192.2 |
| | (n = 43, ±72.8) | | (n = 36, ±79.1) |
| 3 | 167.5 | 3 | 172.5 |
| | (n = 7, ±21.9) | | (n = 10, ±21.9) |

As a result, chronic hepatitis patients showed higher blood βig-h3 concentration than normal people and βig-h3 concentration of lower stage and grade (1 and 2) was confirmed to be higher than that of higher stage and grade (3). Condition of a patient in grade 3 and stage 3 is that hepatocirrhosis has been developed seriously and its activity went through the peak already. Meanwhile, a patient in grade 1 and 2 and stage 1 and 2 shows the condition that inflammatory reaction is developing very actively. Thus, βig-h3 concentration implies the activity of hepatocirrhosis, so that the development of hepatocirrhosis can be observed by measuring blood βig-h3 concentration regularly.

### Example 6

### Relationship Between Rheumatoid Arthritis and βig-h3 Expression

The present inventors confirmed the correlation between rheumatoid arthritis and βig-h3 expression by measuring βig-h3 amount in synovial fluids of patients with osteoarthritis and rheumatoid arthritis with the same method of Example <4-1> (Table 6).

TABLE 6
Concentrations of β ig-h3 in synovial fluids

| | β ig-h3 (ng/mg) |
|---|---|
| Osteoarthritis | 11.0 (n = 29, ±0.3) |
| Rheumatoid arthritis | 21.0 (n = 20, ±2.5) |

As a result, two-fold higher βig-h3 concentration in synovial fluid of rheumatoid arthritis patients was observed, suggesting that measuring βig-h3 concentration in synovial fluid can be an effective way to diagnose osteoarthritis and rheumatoid arthritis.

### Example 7

### Relationship Between Cardiovascular Diseases and βig-h3 Expression

<7-1> Measurement of βig-h3 in Damaged Blood Vessels of Diabetes-Induced Mice

The present inventors investigated the expression patterns of βig-h3 in normal and damaged blood vessels of diabetic mice by immunohistochemical methods in order to confirm the relation between the expression of βig-h3 and cardiovascular diseases.

As a result, βig-h3 protein was expressed much greatly in damaged blood vessels of diabetic mice than in normal blood vessels (FIG. 18).

<7-2> Measurement of βig-h3 Expression Induced by TGF-β in Vascular Smooth Muscle Cells

Based on that βig-h3 expression is induced by TGF-β that plays an important role in the development of vascular diseases, the present inventors tried to confirm the correlation βig-h3 expression and cardiovascular diseases. Particularly, the present inventors measured the expression pattern of βig-h3 induced by TGF-β1 in vascular smooth muscle cells forming blood vessels with the same method of Example <4-1>.

As a result, it was confirmed that βig-h3 expression increases as the amount of TGF-β1 increases (FIG. 19).

From the above results, it was confirmed that the expression of βig-h3 in blood and tissues reflects the damage of them. Therefore, the method for measuring the amount of βig-h3 protein of the present invention can be effectively used for the diagnosis of various cardiovascular diseases.

### INDUSTRIAL APPLICABILITY

As described hereinbefore, the method for measuring the amount of βig-h3 protein of the present invention in which human βig-h3, mouse βig-h3, βig-h3 D-IV(1×) or βig-h3 D-IV(4×) are used as a standard protein is inexpensive and very accurate in measuring βig-h3 concentration in various body fluids. The amount of βig-h3 sensitively reflects TGF-β related diseases such as renal diseases, hepatic diseases, rheumatoid arthritis and cardiovascular diseases in the early stages, so that the method of the present invention can be effectively used for the examination of the damage and the progress of those diseases and for the diagnosis thereof.

1-16. (canceled)

17. A method for diagnosing renal diseases, hepatic diseases, rheumatoid arthritis or cardiovascular diseases, said method comprising detecting an amount of βig-h3 protein comprising the following steps:
(a) preparing recombinant proteins of βig-h3 or βig-h3 fas-1 domains, their fragments or derivatives, as standard proteins;
(b) preparing specific ligands against the above recombinant proteins, their fragments or derivatives of the above step 1; and
(c) measuring the amount of βig-h3 protein of samples with the method using binding reaction of ligands of the above step 2 with the recombinant proteins, their fragments or derivatives of the above step 1.

18. The method as set forth in claim 17, wherein the ligands of step 1) are selected from a group consisting of antibodies, RNA, DNA, lipids, proteins, organic compounds and inorganic compounds.

19. The method as set forth in claim 17, wherein the specific binding reaction of step 3) is antigen-antibody reaction.

20. The method as set forth in claim 19, wherein the antigen-antibody reaction is performed by a method selected from a group consisting of immunoblotting, immunoprecipitation, ELISA, RIA, protein chip, rapid assay and microarray.

21. The method as set forth in claim 19, wherein the antigen-antibody reaction of step 3) comprises the following steps:
(a) coating recombinant proteins of βig-h3 or βig-h3 fas-1 domains, their fragments or derivatives to matrix;
(b) reacting antibody against the protein of the above step 1, its fragments or derivatives with sample;
(c) adding the reactant of the above step 2 to the coated protein of step 1 and waiting for reaction, and then washing thereof; and
(d) adding the secondary antibody to the reactant of the above step 3 for further reaction, and then measuring OD.

22. The method as set forth in claim 17, wherein the βig-h3 protein is human βig-h3 protein having an amino acid sequence represented by SEQ ID NO:3 or mouse βig-h3 protein having an amino acid sequence represented by SEQ ID NO:5.

23. The method as set forth in claim 17, wherein the recombinant βig-h3 proteins comprising 4^{th} fas-1 domains have 1-10 repeatedly-linked fas-1 domains.

24. The method as set forth in claim 23, wherein the fas-1 domain of βig-h3 is selected from a group consisting of sequences represented by SEQ ID NO:7, SEQ ID NO:8, SEQ ID NO:9 and SEQ ID NO:10.

25. The method as set forth in claim 17, wherein the sample can be any body fluid including urine, blood or synovial fluid.

26. A diagnostic kit for the renal diseases, hepatic diseases, rheumatoid arthritis or cardiovascular diseases comprising βig-h3 protein or recombinant proteins of fas-1 domain in the βig-h3 protein or fragments or derivatives thereof and their ligands.

27. The diagnostic kit as set forth in claim 26, wherein the ligand is selected from a group consisting of antibody specifically binding to βig-h3 protein, fas-1 domain of βig-h3, their fragments or derivatives, RNA, DNA, lipids, proteins, organic compounds and inorganic compounds.

28. The diagnostic kit as set forth in claim 27, wherein the ligand is antibody.

29. The diagnostic kit as set forth in claim 28, wherein the kit additionally includes buffer solution, secondary antibody, washing solution, stop solution or coloring substrate.

30. The diagnostic kit as set forth in claim 26, wherein the βig-h3 protein is human βig-h3 protein having an amino acid sequence represented by SEQ ID NO:3 or mouse βig-h3 protein having an amino acid sequence represented by SEQ ID NO:5.

31. The diagnostic kit as set forth in claim 26, wherein 1 or 2-10 4^{th} fas-1 domains of βig-h3 protein are repeatedly linked.

32. The diagnostic kit as set forth in claim 31, wherein the fas-1 domain of βig-h3 is selected from a group consisting of sequences represented by SEQ ID NO:7, SEQ ID NO:8, SEQ ID NO:9 and SEQ ID NO:10.
